(19) United States
(12) Patent Application Publication
(10) Pub. No.: US 2009/0216018 A1
(43) Pub. Date: Aug. 27, 2009

(54) ORGANOMETALLIC COMPLEXES

(75) Inventors: Norman Herron, Newark, DE (US); Eric Smith, Hockession, DE (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: E.I. du Pont de Nemours, Wilmington, DE (US)

(21) Appl. No.: 11/722,129

(22) PCT Filed: Dec. 29, 2005
(86) PCT No.: PCT/US05/47501

§ 371 Date: Jan. 31, 2008

Related U.S. Application Data
(60) Provisional application No. 60/640,493, filed on Dec. 30, 2004.
(60) Provisional application No. 60/694,943, filed on Jun. 28, 2005.

(51) Int. Cl. C07F 5/06; C07C 255/50

(52) U.S. Cl. 546/7; 558/411

(57) ABSTRACT

Organometallic complexes are provided, methods for making the same, and their use in devices and sub-assemblies.

### CROSS REFERENCE

This application claims benefit to U.S. Provisional Application Ser. Nos. 60/640,493, filed Dec. 30, 2004 and 60/694,943, filed Jun. 28, 2005, the disclosures of which are each incorporated herein by reference in their entireties.

### FIELD

This disclosure relates generally to organometallic complexes, for example, those found in organic electronic devices, and materials and methods for fabrication of the same.

### BACKGROUND

Organic electronic devices convert electrical energy into radiation, detect signals through electronic processes, convert radiation into electrical energy, or include one or more organic semiconductor layers. An organic light-emitting diode (OLED) is an organic electronic device comprising an organic layer capable of electroluminescence. In some OLEDs, these photoactive organic layers comprise simple organic molecules, conjugated polymers, or organometallic complexes.

As can be appreciated, it is important to develop organometallic complexes.

### SUMMARY

Organometallic complexes are provided, and methods for making, and devices and sub-assemblies including, the same.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 is a schematic diagram of an organic electronic device.

The figures are provided by way of example and are not intended to limit the invention. Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Organometallic complexes are provided having at least one ligand having Formula I or II:

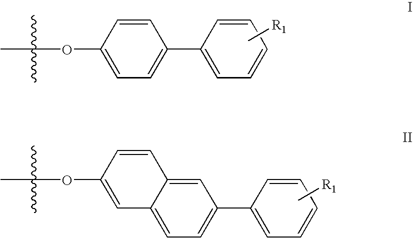

wherein R₁ is alkyl or aryl. The bond crossed by the wavy line indicates a bond to the metal.

In one embodiment, R₁ is in the para-position.

In one embodiment, R₁ is an alkyl group. In one embodiment, R₁ is a C4-C5 alkyl. In one embodiment, R₁ is t-butyl.

In one embodiment, R₁ is aryl. In one embodiment, R₁ is phenyl.

It is understood that R₁ can be further substituted with any substituent that increases the solubility of the ligand in a non-polar solvent.

In one embodiment, an organometallic complex is provided comprising a formula:

[Y]ₙMZ

wherein:

n is 1, 2, or 3;

M is a metal in a +2, +3, or +4 oxidation state;

Y is a ligand comprising 8-hydroxyquinoline or alkyl-substituted 8-hydroxyquinoline at each occurrence; and

Z is a ligand of Formula I or II as described above.

In one embodiment, M is Al, Zn, Zr, or Ga. In one embodiment, M is Al.

In one embodiment, the alkyl-substituted 8-hydroxyquinoline is 2-alkyl-8-hydroxyquinoline. In one embodiment, the alkyl-substituted 8-hydroxyquinoline is 2-methyl-8-hydroxyquinoline.

In one embodiment, the organometallic complex is electroluminescent.

In one embodiment, there is provided an organometallic complex having the formula:

MYₙZ

wherein:

n is 1, 2, or 3;

M is a metal in a +2, +3, or +4 oxidation state;

Y is selected from 8-hydroxyquinolate and substituted 8-hydroxyquinolate; and

Z is a compound of Formula III or IV:

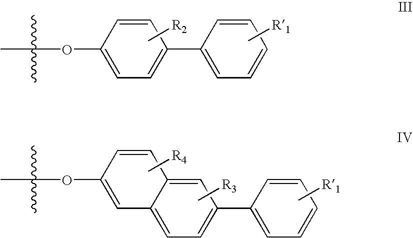

wherein:

R′₁ is one or more solvent-solubilizing or Tg enhancing groups;

R₂, R₃, and R₄ are independently one or more selected from the group consisting of H, alkyl, substituted alkyl, aryl, substituted aryl, F, CN, a solvent-solubilizing group, and a Tg enhancing group.

In one embodiment, M is Al, Zn, Zr, In or Ga. In one embodiment, M is Al.

In one embodiment, R₁ is an alkyl group. In one embodiment, R₁ is a C1-C6 alkyl. In one embodiment, R₁ is cyano, alkyl, fluoroalkyl, aryl, fluoroaryl, alkylaryl, alkoxy, aryloxy, fluoroalkoxy, or fluoroaryloxy, or their hetero-analogs. In one embodiment, R₁ is phenyl, fluorophenyl, alkylphenyl, fluoroalkylphenyl, alkoxy phenyl, or fluoroalkoxyphenyl.

In one embodiment, all R₂, R₃, and R₄ are H.

As used herein, the term “solvent-solubilizing” indicates that the solubility or dispersability of a material in at least one organic solvent has been increased over that of the standard parent molecules, BAlQ, (MY₂Z where Y=2-methyl-8-hydroxyquinolinate and Z=4-phenylphenol) about 1.9 wt % in toluene, or BAlnapQ (MY₂Z where Y=2-methyl-8-hydroxyquinolinate and Z=6-phenyl-naphth-2-ol) about 1.3 wt % in toluene, all at room temperature. The term “Tg enhancing” indicates that the glass transition temperature of the material has been raised over that of the standard parent molecules, BAlQ 103° C. and BAlnapQ 112° C.

The term “substituted 8-hydroxyquinolate” indicates 8-hydroxyquinolate having at least one alkyl, aryl, substituted alkyl, or substituted aryl substituent.

The prefix “hetero” indicates that one or more carbon atoms have been replaced with a different atom. The prefix “fluoro” indicates that one or more hydrogen atoms have been replaced with a fluorine atom.

As used herein, “Y” and “Z” are intended to mean ligands on a metal complex.

Any number of other configurations for the containment structure are contemplated.

In one embodiment, compositions are provided comprising the above-described compounds and at least one solvent, processing aid, charge transporting material, or charge blocking material. These compositions can be in any form, including, but not limited to solvents, emulsions, and colloidal dispersions.

### Device

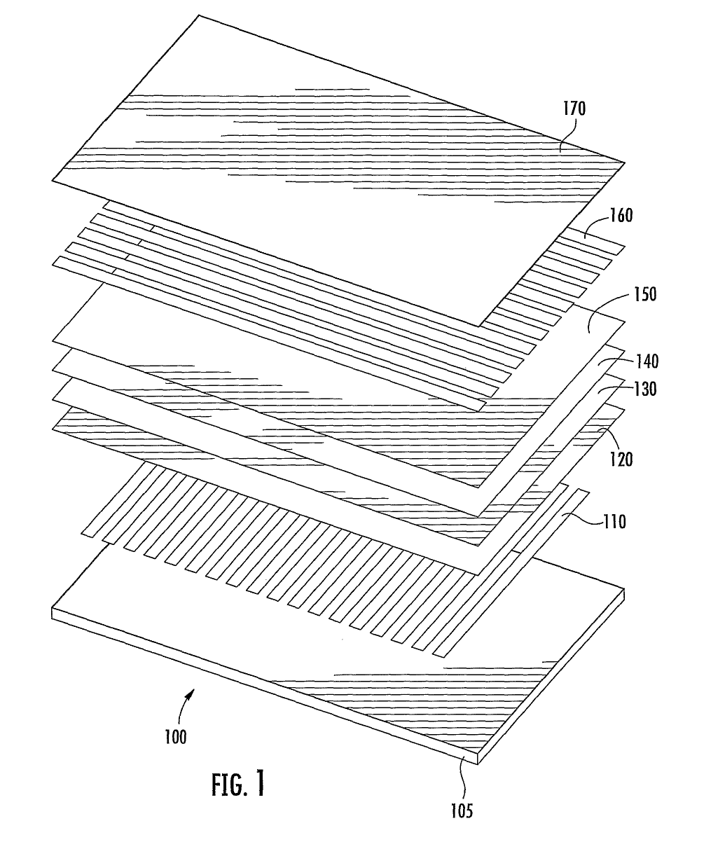

Referring to FIG. 1, an exemplary organic electronic device 100 is shown. The device 100 includes a substrate 105. The substrate 105 may be rigid or flexible, for example, glass, ceramic, metal, or plastic. When voltage is applied, emitted light is visible through the substrate 105.

A first electrical contact layer 110 is deposited on the substrate 105. For illustrative purposes, the layer 110 is an anode layer. Anode layers may be deposited as lines. The anode can be made of, for example, materials containing or comprising metal, mixed metals, alloy, metal oxides or mixed-metal oxide. The anode may comprise a conducting polymer, polymer blend or polymer mixtures. Suitable metals include the Group 11 metals, the metals in Groups 4, 5, and 6, and the Group 8, 10 transition metals. If the anode is to be light-transmitting, mixed-metal oxides of Groups 12, 13 and 14 metals, such as indium-tin-oxide, are generally used. The anode may also comprise an organic material, especially a conducting polymer such as polyaniline, including exemplary materials as described in Flexible Light-Emitting Diodes Made From Soluble Conducting Polymer, Nature 1992, 357, 477-479. At least one of the anode and cathode should be at least partially transparent to allow the generated light to be observed.

An optional buffer layer 120, such as hole transport materials, may be deposited over the anode layer 110, the latter being sometimes referred to as the “hole-injecting contact layer.” Examples of hole transport materials suitable for use as the layer 120 have been summarized, for example, in Kirk Othmer, Encyclopedia of Chemical Technology, Vol. 18, 837-860 (4^{th} ed. 1996). Both hole transporting “small” molecules as well as oligomers and polymers may be used. Hole transporting molecules include, but are not limited to: N,N′ diphenyl-N,N′-bis(3-methylphenyl)-[1,1′-biphenyl]-4,4′-diamine (TPD), 1,1 bis[(di-4-tolylamino) phenyl]cyclohexane (TAPC), N,N′ bis(4-methylphenyl)-N,N′-bis(4-ethylphenyl)-[1,1′-(3,3′-dimethyl)biphenyl]-4,4′-diamine (ETPD), tetrakis (3-methylphenyl)-N,N,N′,N′-2,5-phenylenediamine (PDA), a-phenyl 4-N,N-diphenylaminostyrene (TPS), p (diethylamino)benzaldehyde diphenylhydrazone (DEH), triphenylamine (TPA), bis[4 (N,N-diethylamino)-2-methylphenyl](4-methylphenyl)methane (MPMP), 1 phenyl-3-[p-(diethylamino)styryl]-5-[p-(diethylamino)phenyl]pyrazoline (PPR or DEASP), 1,2 trans-bis(9H-carbazol-9-yl)cyclobutane (DCZB), N,N,N′,N′ tetrakis(4-methylphenyl)-(1,1′-biphenyl)-4,4′-diamine (TTB); 4,4′, N,N′, dicarbazolyl-biphenyl (CBP); and porphyrinic compounds, such as copper phthalocyanine. Useful hole transporting polymers include, but are not limited to, polyvinylcarbazole, (phenylmethyl)polysilane, and polyaniline. Conducting polymers are useful as a class. It is also possible to obtain hole transporting polymers by doping hole transporting moieties, such as those mentioned above, into polymers such as polystyrenes and polycarbonates.

An organic layer 130 may be deposited over the buffer layer 120 when present, or over the first electrical contact layer 110. In some embodiments, the organic layer 130 may be a number of discrete layers comprising a variety of components. Depending upon the application of the device, the organic layer 130 can be a light-emitting layer that is activated by an applied voltage (such as in a light-emitting diode or light-emitting electrochemical cell), or a layer of material that responds to radiant energy and generates a signal with or without an applied bias voltage (such as in a photodetector).

Other layers in the device can be made of any materials which are known to be useful in such layers upon consideration of the function to be served by such layers.

Any organic electroluminescent (“EL”) material can be used as a photoactive material (e.g., in layer 130). Such materials include, but are not limited to, fluorescent dyes, small molecule organic fluorescent compounds, fluorescent and phosphorescent metal complexes, conjugated polymers, and mixtures thereof. Examples of fluorescent dyes include, but are not limited to, pyrene, perylene, rubrene, derivatives thereof, and mixtures thereof. Examples of metal complexes include, but are not limited to, metal chelated oxinoid compounds, such as tris(8-hydroxyquinolato)aluminum (Alq3); cyclometallated iridium and platinum electroluminescent compounds, such as complexes of Iridium with phenylpyridine, phenylquinoline, or phenylpyrimidine ligands as disclosed in Petrov et al., Published PCT Application WO 02/02714, and organometallic complexes described in, for example, published applications US 2001/0019782, EP 1191612, WO 02/15645, and EP 1191614; and mixtures thereof. Electroluminescent emissive layers comprising a charge carrying host material and a metal complex have been described by Thompson et al., in U.S. Pat. No. 6,303,238, and by Burrows and Thompson in published PCT applications WO 00/70655 and WO 01/41512. Examples of conjugated polymers include, but are not limited to poly(phenylenevinylenes), polyfluorenes, poly(spirobifluorenes), polythiophenes, poly(p-phenylenes), copolymers thereof, and mixtures thereof.

In one embodiment of the devices, the photoactive material can be an organometallic complex. In another embodiment, the photoactive material is a cyclometallated complex of iridium or platinum. Other useful photoactive materials may be employed as well. Complexes of iridium with phenylpyridine, phenylquinoline, or phenylpyrimidine ligands have been disclosed as electroluminescent compounds in Petrov et al., Published PCT Application WO 02/02714. Other organometallic complexes have been described in, for example, published applications US 2001/0019782, EP 1191612, WO 02/15645, and EP 1191614. Electroluminescent devices with an active layer of polyvinyl carbazole (PVK) doped with metallic complexes of iridium have been described by Burrows and Thompson in published PCT applications WO 00/70655 and WO 01/41512. Electroluminescent emissive layers comprising a charge carrying host material and a phosphorescent platinum complex have been described by Thompson et al., in U.S. Pat. No. 6,303,238, Bradley et al., in Synth. Met. 2001, 116 (1-3), 379-383, and Campbell et al., in Phys. Rev. B, Vol. 65 085210.

A second electrical contact layer 160 is deposited on the organic layer 130. For illustrative purposes, the layer 160 is a cathode layer.

Cathode layers may be deposited as lines or as a film. The cathode can be any metal or nonmetal having a lower work function than the anode. Exemplary materials for the cathode can include alkali metals, especially lithium, the Group 2 (alkaline earth) metals, the Group 12 metals, including the rare earth elements and lanthanides, and the actinides. Materials such as aluminum, indium, calcium, barium, samarium and magnesium, as well as combinations, can be used. Lithium-containing and other compounds, such as LiF and Li₂O, may also be deposited between an organic layer and the cathode layer to lower the operating voltage of the system.

An electron transport layer 140 or electron injection layer 150 is optionally disposed adjacent to the cathode, the cathode being sometimes referred to as the “electron-injecting contact layer.”

An encapsulation layer 170 is deposited over the contact layer 160 to prevent entry of undesirable components, such as water and oxygen, into the device 100. Such components can have a deleterious effect on the organic layer 130. In one embodiment, the encapsulation layer 170 is a barrier layer or film.

Though not depicted, it is understood that the device 100 may comprise additional layers. For example, there can be a layer (not shown) between the anode 110 and hole transport layer 120 to facilitate positive charge transport and/or band-gap matching of the layers, or to function as a protective layer. Other layers that are known in the art or otherwise may be used. In addition, any of the above-described layers may comprise two or more sub-layers or may form a laminar structure. Alternatively, some or all of anode layer 110 the hole transport layer 120, the electron transport layers 140 and 150, cathode layer 160, and other layers may be treated, especially surface treated, to increase charge carrier transport efficiency or other physical properties of the devices. The choice of materials for each of the component layers is preferably determined by balancing the goals of providing a device with high device efficiency with device operational lifetime considerations, fabrication time and complexity factors and other considerations appreciated by persons skilled in the art. It will be appreciated that determining optimal components, component configurations, and compositional identities would be routine to those of ordinary skill of in the art.

In one embodiment, the different layers have the following range of thicknesses: anode 110, 500-5000 Å, in one embodiment 1000-2000 Å; hole transport layer 120, 50-2000 Å, in one embodiment 200-1000 Å; photoactive layer 130, 10-2000 Å, in one embodiment 100-1000 Å; layers 140 and 150, 50-2000 Å, in one embodiment 100-1000 Å; cathode 160, 200-10000 Å, in one embodiment 300-5000 Å. The location of the electron-hole recombination zone in the device, and thus the emission spectrum of the device, can be affected by the relative thickness of each layer. Thus the thickness of the electron-transport layer should be chosen so that the electron-hole recombination zone is in the light-emitting layer. The desired ratio of layer thicknesses will depend on the exact nature of the materials used.

In one embodiment, the device has the following structure, in order: anode, buffer layer, hole transport layer, photoactive layer, electron transport layer, electron injection layer, cathode. In one embodiment, the anode is made of indium tin oxide or indium zinc oxide. In one embodiment, the buffer layer comprises a conducting polymer selected from the group consisting of polythiophenes, polyanilines, polypyrroles, copolymers thereof, and mixtures thereof. In one embodiment, the buffer layer comprises a complex of a conducting polymer and a colloid-forming polymeric acid. In one embodiment, the buffer layer comprises a compound having triarylamine or triarylmethane groups. In one embodiment, the buffer layer comprises a material selected from the group consisting of TPD, MPMP, NPB, CBP, and mixtures thereof, as defined above.

In one embodiment, the hole transport layer comprises polymeric hole transport material. In one embodiment, the hole transport layer is crosslinkable. In one embodiment, the hole transport layer comprises a compound having triarylamine or triarylmethane groups. In one embodiment, the buffer layer comprises a material selected from the group consisting of TPD, MPMP, NPB, CBP, and mixtures thereof, as defined above.

In one embodiment, the photoactive layer comprises an electroluminescent metal complex and a host material. The host can be a charge transport material. In one embodiment, the host material is an organometallic complex having the formula MYnZ, as defined herein. In one embodiment, the electroluminescent complex is present in an amount of at least 1% by weight. In one embodiment, the electroluminescent complex is 2-20% by weight. In one embodiment, the electroluminescent complex is 20-50% by weight. In one embodiment, the electroluminescent complex is 50-80% by weight. In one embodiment, the electroluminescent complex is 80-99% by weight. In one embodiment, the metal complex is a cyclometallated complex of iridium, platinum, rhenium, or osmium. In one embodiment, the photoactive layer further comprises a second host material. The second host can be a charge transport material. In one embodiment, the second host is a hole transport material. In one embodiment, the second host is an electron transport material. In one embodiment, the second host material is a metal complex of a hydroxyaryl-N-heterocycle. In one embodiment, the hydroxyaryl-N-heterocycle is unsubstituted or substituted 8-hydroxyquinoline. In one embodiment, the metal is aluminum. In one embodiment, the second host is a material selected from the group consisting of tris(8-hydroxyquinolinato)aluminum, bis(8-hydroxyquinolinato)(4-phenylphenolato)aluminum, tetrakis(8-hydroxyquinolinato)zirconium, and mixtures thereof. The ratio of the first host to the second host can be 1:100 to 100:1. In one embodiment the ratio is from 1:10 to 10:1. In one embodiment, the ratio is from 1:10 to 1:5. In one embodiment, the ratio is from 1:5 to 1:1. In one embodiment, the ratio is from 1:1 to 5:1. In one embodiment, the ratio is from 5:1 to 5:10.

In one embodiment, the electron transport layer comprises a metal complex of a hydroxyaryl-N-heterocycle. In one embodiment, the hydroxyaryl-N-heterocycle is unsubstituted or substituted 8-hydroxyquinoline. In one embodiment, the metal is aluminum. In one embodiment, the electron transport layer comprises a material selected from the group consisting of tris(8-hydroxyquinolinato)aluminum, bis(8-hydroxyquinolinato)(4-phenylphenolato)aluminum, tetrakis(8-hydroxyquinolinato)zirconium, and mixtures thereof. In one embodiment, the electron injection layer is LiF or LiO2. In one embodiment, the cathode is Al or Ba/Al.

In one embodiment, the device is fabricated by liquid deposition of the buffer layer, the hole transport layer, and the photoactive layer, and by vapor deposition of the electron transport layer, the electron injection layer, and the cathode.

The buffer layer can be deposited from any liquid medium in which it is dissolved or dispersed and from which it will form a film. In one embodiment, the liquid medium consists essentially of one or more organic solvents. In one embodiment, the liquid medium consists essentially of water or water and an organic solvent. In one embodiment the organic solvent is selected from the group consisting of alcohols, ketones, cyclic ethers, and polyols. In one embodiment, the organic liquid is selected from dimethylacetamide (“DMAc”), N methylpyrrolidone (“NMP”), dimethylformamide (“DMF”), ethylene glycol (“EG”), aliphatic alcohols, and mixtures thereof. The buffer material can be present in the liquid medium in an amount from 0.5 to 10 percent by weight. Other weight percentages of buffer material may be used depending upon the liquid medium. The buffer layer can be applied by any continuous or discontinuous liquid deposition technique. In one embodiment, the buffer layer is applied by spin coating. In one embodiment, the buffer layer is applied by ink jet printing. After liquid deposition, the liquid medium can be removed in air, in an inert atmosphere, or by vacuum, at room temperature or with heating. In one embodiment, the layer is heated to a temperature less than 275° C. In one embodiment, the heating temperature is between 100° C. and 275° C. In one embodiment, the heating temperature is between 100° C. and 120° C. In one embodiment, the heating temperature is between 120° C. and 140° C. In one embodiment, the heating temperature is between 140° C. and 160° C. In one embodiment, the heating temperature is between 160° C. and 180° C. In one embodiment, the heating temperature is between 180° C. and 200° C. In one embodiment, the heating temperature is between 200° C. and 220° C. In one embodiment, the heating temperature is between 190° C. and 220° C. In one embodiment, the heating temperature is between 220° C. and 240° C. In one embodiment, the heating temperature is between 240° C. and 260° C. In one embodiment, the heating temperature is between 260° C. and 275° C. The heating time is dependent upon the temperature, and is generally between 5 and 60 minutes. In one embodiment, the final layer thickness is between 5 and 200 nm. In one embodiment, the final layer thickness is between 5 and 40 nm. In one embodiment, the final layer thickness is between 40 and 80 nm. In one embodiment, the final layer thickness is between 80 and 120 nm. In one embodiment, the final layer thickness is between 120 and 160 nm. In one embodiment, the final layer thickness is between 160 and 200 nm.

The hole transport layer can be deposited from any liquid medium in which it is dissolved or dispersed and from which it will form a film. In one embodiment, the liquid medium consists essentially of one or more organic solvents. In one embodiment, the liquid medium consists essentially of water or water and an organic solvent. In one embodiment the organic solvent is an aromatic solvent. In one embodiment, the organic liquid is selected from chloroform, dichloromethane, toluene, anisole, and mixtures thereof. The hole transport material can be present in the liquid medium in a concentration of 0.2 to 2 percent by weight. Other weight percentages of hole transport material may be used depending upon the liquid medium. The hole transport layer can be applied by any continuous or discontinuous liquid deposition technique. In one embodiment, the hole transport layer is applied by spin coating. In one embodiment, the hole transport layer is applied by ink jet printing. After liquid deposition, the liquid medium can be removed in air, in an inert atmosphere, or by vacuum, at room temperature or with heating. In one embodiment, the layer is heated to a temperature less than 275° C. In one embodiment, the heating temperature is between 170° C. and 275° C. In one embodiment, the heating temperature is between 170° C. and 200° C. In one embodiment, the heating temperature is between 190° C. and 220° C. In one embodiment, the heating temperature is between 210° C. and 240° C. In one embodiment, the heating temperature is between 230° C. and 270° C. The heating time is dependent upon the temperature, and is generally between 5 and 60 minutes. In one embodiment, the final layer thickness is between 5 and 50 nm. In one embodiment, the final layer thickness is between 5 and 15 nm. In one embodiment, the final layer thickness is between 15 and 25 nm. In one embodiment, the final layer thickness is between 25 and 35 nm. In one embodiment, the final layer thickness is between 35 and 50 nm.

The photoactive layer can be deposited from any liquid medium in which it is dissolved or dispersed and from which it will form a film. In one embodiment, the liquid medium consists essentially of one or more organic solvents. In one embodiment, the liquid medium consists essentially of water or water and an organic solvent. In one embodiment the organic solvent is an aromatic solvent. In one embodiment, the organic liquid is selected from chloroform, dichloromethane, toluene, anisole, and mixtures thereof. The photoactive material can be present in the liquid medium in a concentration of 0.2 to 2 percent by weight. Other weight percentages of photoactive material may be used depending upon the liquid medium. The photoactive layer can be applied by any continuous or discontinuous liquid deposition technique. In one embodiment, the photoactive layer is applied by spin coating. In one embodiment, the photoactive layer is applied by ink jet printing. After liquid deposition, the liquid medium can be removed in air, in an inert atmosphere, or by vacuum, at room temperature or with heating. In one embodiment, the deposited layer is heated to a temperature that is less than the Tg of the material having the lowest Tg. In one embodiment, the heating temperature is at least 10° C. less than the lowest Tg. In one embodiment, the heating temperature is at least 20° C. less than the lowest Tg. In one embodiment, the heating temperature is at least 30° C. less than the lowest Tg. In one embodiment, the heating temperature is between 50° C. and 150° C. In one embodiment, the heating temperature is between 50° C. and 75° C. In one embodiment, the heating temperature is between 75° C. and 100° C. In one embodiment, the heating temperature is between 100° C. and 125° C. In one embodiment, the heating temperature is between 125° C. and 150° C. The heating time is dependent upon the temperature, and is generally between 5 and 60 minutes. In one embodiment, the final layer thickness is between 25 and 100 nm. In one embodiment, the final layer thickness is between 25 and 40 nm. In one embodiment, the final layer thickness is between 40 and 65 nm. In one embodiment, the final layer thickness is between 65 and 80 nm. In one embodiment, the final layer thickness is between 80 and 100 nm.

The electron transport layer can be deposited by any vapor deposition method. In one embodiment, it is deposited by thermal evaporation under vacuum. In one embodiment, the final layer thickness is between 1 and 100 nm. In one embodiment, the final layer thickness is between 1 and 15 nm. In one embodiment, the final layer thickness is between 15 and 30 nm. In one embodiment, the final layer thickness is between 30 and 45 nm. In one embodiment, the final layer thickness is between 45 and 60 nm. In one embodiment, the final layer thickness is between 60 and 75 nm. In one embodiment, the final layer thickness is between 75 and 90 nm. In one embodiment, the final layer thickness is between 90 and 100 nm.

The electron injection layer can be deposited by any vapor deposition method. In one embodiment, it is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of 1 to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 0.1 and 3 nm. In one embodiment, the final layer thickness is between 0.1 and 1 nm. In one embodiment, the final layer thickness is between 1 and 2 nm. In one embodiment, the final layer thickness is between 2 and 3 nm.

The cathode can be deposited by any vapor deposition method. In one embodiment, it is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of 1 to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 10 and 10000 nm. In one embodiment, the final layer thickness is between 10 and 1000 nm. In one embodiment, the final layer thickness is between 10 and 50 nm. In one embodiment, the final layer thickness is between 50 and 100 nm. In one embodiment, the final layer thickness is between 100 and 200 nm. In one embodiment, the final layer thickness is between 200 and 300 nm. In one embodiment, the final layer thickness is between 300 and 400 nm. In one embodiment, the final layer thickness is between 400 and 500 nm. In one embodiment, the final layer thickness is between 500 and 600 nm. In one embodiment, the final layer thickness is between 600 and 700 nm. In one embodiment, the final layer thickness is between 700 and 800 nm. In one embodiment, the final layer thickness is between 800 and 900 nm. In one embodiment, the final layer thickness is between 900 and 1000 nm. In one embodiment, the final layer thickness is between 1000 and 2000 nm. In one embodiment, the final layer thickness is between 2000 and 3000 nm. In one embodiment, the final layer thickness is between 3000 and 4000 nm. In one embodiment, the final layer thickness is between 4000 and 5000 nm. In one embodiment, the final layer thickness is between 5000 and 6000 nm. In one embodiment, the final layer thickness is between 6000 and 7000 nm. In one embodiment, the final layer thickness is between 7000 and 8000 nm. In one embodiment, the final layer thickness is between 8000 and 9000 nm. In one embodiment, the final layer thickness is between 9000 and 10000 nm.

In one embodiment, the device is fabricated by vapor deposition of the buffer layer, the hole transport layer, and the photoactive layer, the electron transport layer, the electron injection layer, and the cathode.

In one embodiment, the buffer layer is applied by vapor deposition. In one embodiment, it is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of 1 to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 5 and 200 nm. In one embodiment, the final layer thickness is between 5 and 30 nm. In one embodiment, the final layer thickness is between 30 and 60 nm. In one embodiment, the final layer thickness is between 60 and 90 nm. In one embodiment, the final layer thickness is between 90 and 120 nm. In one embodiment, the final layer thickness is between 120 and 150 nm. In one embodiment, the final layer thickness is between 150 and 280 nm. In one embodiment, the final layer thickness is between 180 and 200 nm.

In one embodiment, the hole transport layer is applied by vapor deposition. In one embodiment, it is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 5 and 200 nm. In one embodiment, the final layer thickness is between 5 and 30 nm. In one embodiment, the final layer thickness is between 30 and 60 nm. In one embodiment, the final layer thickness is between 60 and 90 nm. In one embodiment, the final layer thickness is between 90 and 120 nm. In one embodiment, the final layer thickness is between 120 and 150 nm. In one embodiment, the final layer thickness is between 150 and 280 nm. In one embodiment, the final layer thickness is between 180 and 200 nm.

In one embodiment, the photoactive layer is applied by vapor deposition. In one embodiment, it is deposited by thermal evaporation under vacuum. In one embodiment, the photoactive layer consists essentially of a single electroluminescent compound, which is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of 1 to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 5 and 200 nm. In one embodiment, the final layer thickness is between 5 and 30 nm. In one embodiment, the final layer thickness is between 30 and 60 nm. In one embodiment, the final layer thickness is between 60 and 90 nm. In one embodiment, the final layer thickness is between 90 and 120 nm. In one embodiment, the final layer thickness is between 120 and 150 nm. In one embodiment, the final layer thickness is between 150 and 280 nm. In one embodiment, the final layer thickness is between 180 and 200 nm.

In one embodiment, the photoactive layer comprises two electroluminescent materials, each of which is applied by thermal evaporation under vacuum. Any of the above listed vacuum conditions and temperatures can be used. Any of the above listed deposition rates can be used. The relative deposition rates can be from 50:1 to 1:50. In one embodiment, the relative deposition rates are from 1:1 to 1:3. In one embodiment, the relative deposition rates are from 1:3 to 1:5. In one embodiment, the relative deposition rates are from 1:5 to 1:8. In one embodiment, the relative deposition rates are from 1:8 to 1:10. In one embodiment, the relative deposition rates are from 1:10 to 1:20. In one embodiment, the relative deposition rates are from 1:20 to 1:30. In one embodiment, the relative deposition rates are from 1:30 to 1:50. The total thickness of the layer can be the same as that described above for a single-component photoactive layer.

In one embodiment, the photoactive layer comprises one electroluminescent material and at least one host material, each of which is applied by thermal evaporation under vacuum. Any of the above listed vacuum conditions and temperatures can be used. Any of the above listed deposition rates can be used. The relative deposition rate of electroluminescent material to host can be from 1:1 to 1:99. In one embodiment, the relative deposition rates are from 1:1 to 1:3. In one embodiment, the relative deposition rates are from 1:3 to 1:5. In one embodiment, the relative deposition rates are from 1:5 to 1:8. In one embodiment, the relative deposition rates are from 1:8 to 1:10. In one embodiment, the relative deposition rates are from 1:10 to 1:20. In one embodiment, the relative deposition rates are from 1:20 to 1:30. In one embodiment, the relative deposition rates are from 1:30 to 1:40. In one embodiment, the relative deposition rates are from 1:40 to 1:50. In one embodiment, the relative deposition rates are from 1:50 to 1:60. In one embodiment, the relative deposition rates are from 1:60 to 1:70. In one embodiment, the relative deposition rates are from 1:70 to 1:80. In one embodiment, the relative deposition rates are from 1:80 to 1:90. In one embodiment, the relative deposition rates are from 1:90 to 1:99. The total thickness of the layer can be the same as that described above for a single-component photoactive layer.

In one embodiment, the electron transport layer is applied by vapor deposition. In one embodiment, it is deposited by thermal evaporation under vacuum. In one embodiment, the vacuum is less than 10-6 torr. In one embodiment, the vacuum is less than 10-7 torr. In one embodiment, the vacuum is less than 10-8 torr. In one embodiment, the material is heated to a temperature in the range of 100° C. to 400° C.; 150° C. to 350° C. preferably. In one embodiment, the material is deposited at a rate of 0.5 to 10 \acute{Å}/sec. In one embodiment, the material is deposited at a rate of 0.5 to 1 \acute{Å}/sec. In one embodiment, the material is deposited at a rate of 1 to 2 \acute{Å}/sec. In one embodiment, the material is deposited at a rate of 2 to 3 \acute{Å}/sec. In one embodiment, the material is deposited at a rate of 3 to 4 \acute{Å}/sec. In one embodiment, the material is deposited at a rate of 4 to 5 \acute{Å}/sec. In one embodiment, the material is deposited at a rate of 5 to 6 \acute{Å}/sec. In one embodiment, the material is deposited at a rate of 6 to 7 \acute{Å}/sec. In one embodiment, the material is deposited at a rate of 7 to 8 \acute{Å}/sec. In one embodiment, the material is deposited at a rate of 8 to 9 \acute{Å}/sec. In one embodiment, the material is deposited at a rate of 9 to 10 \acute{Å}/sec. In one embodiment, the final layer thickness is between 5 and 200 nm. In one embodiment, the final layer thickness is between 5 and 30 nm. In one embodiment, the final layer thickness is between 30 and 60 nm. In one embodiment, the final layer thickness is between 60 and 90 nm. In one embodiment, the final layer thickness is between 90 and 120 nm. In one embodiment, the final layer thickness is between 120 and 150 nm. In one embodiment, the final layer thickness is between 150 and 280 nm. In one embodiment, the final layer thickness is between 180 and 200 nm.

In one embodiment, the electron injection layer is applied by vapor deposition, as described above.

In one embodiment, the cathode is applied by vapor deposition, as describe above. In one embodiment, the device is fabricated by vapor deposition of some of the organic layers, and liquid deposition of some of the organic layers. In one embodiment, the device is fabricated by liquid deposition of the buffer layer, and vapor deposition of all of the other layers.

In operation, a voltage from an appropriate power supply (not depicted) is applied to the device 100. Current therefore passes across the layers of the device 100. Electrons enter the organic polymer layer, releasing photons. In some OLEDs, called active matrix OLED displays, individual deposits of photoactive organic films may be independently excited by the passage of current, leading to individual pixels of light emission. In some OLEDs, called passive matrix OLED displays, deposits of photoactive organic films may be excited by rows and columns of electrical contact layers.

Devices can be prepared employing a variety of techniques. These include, by way of non-limiting exemplification, vapor deposition techniques and liquid deposition. Devices may also be sub-assembled into separate articles of manufacture that can then be combined to form the device.

### DEFINITIONS

The use of “a” or “an” are employed to describe elements and components of the invention. This is done merely for convenience and to give a general sense of the invention. This description should be read to include one or at least one and the singular also includes the plural unless it is obvious that it is meant otherwise.

The term “active” when referring to a layer or material is intended to mean a layer or material that exhibits electronic or electro-radiative properties. An active layer material may emit radiation or exhibit a change in concentration of electron-hole pairs when receiving radiation. Thus, the term “active material” refers to a material which electronically facilitates the operation of the device. Examples of active materials include, but are not limited to, materials which conduct, inject, transport, or block a charge, where the charge can be either an electron or a hole. Examples of inactive materials include, but are not limited to, planarization materials, insulating materials, and environmental barrier materials.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “layer” is used interchangeably with the term “film” and refers to a coating covering a desired area. The area can be as large as an entire device or a specific functional area such as the actual visual display, or as small as a single sub-pixel. Films can be formed by any conventional deposition technique, including vapor deposition and liquid deposition. Liquid deposition techniques include, but are not limited to, continuous deposition techniques such as spin coating, gravure coating, curtain coating, dip coating, slot-die coating, spray-coating, and continuous nozzle coating; and discontinuous deposition techniques such as ink jet printing, gravure printing, and screen printing.

The term “organic electronic device” is intended to mean a device including one or more semiconductor layers or materials. Organic electronic devices include, but are not limited to: (1) devices that convert electrical energy into radiation (e.g., a light-emitting diode, light emitting diode display, diode laser, or lighting panel), (2) devices that detect signals through electronic processes (e.g., photodetectors photoconductive cells, photoresistors, photoswitches, phototransistors, phototubes, infrared (“IR”) detectors, or biosensors), (3) devices that convert radiation into electrical energy (e.g., a photovoltaic device or solar cell), and (4) devices that include one or more electronic components that include one or more organic semiconductor layers (e.g., a transistor or diode). The term device also includes coating materials for memory storage devices, antistatic films, biosensors, electrochromic devices, solid electrolyte capacitors, energy storage devices such as a rechargeable battery, and electromagnetic shielding applications.

The term “substrate” is intended to mean a workpiece that can be either rigid or flexible and may include one or more layers of one or more materials, which can include, but are not limited to, glass, polymer, metal, or ceramic materials, or combinations thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

To the extent not described herein, many details regarding specific materials, processing acts, and circuits are conventional and may be found in textbooks and other sources within the organic light-emitting diode display, photodetector, photovoltaic, and semiconductive member arts.

### EXAMPLES

The concepts described herein will be further described in the following examples, which do not limit the scope of the invention described in the claims.

### Example 1 Preparation of an Aluminum Complex of 4-(4-t-butylphenyl)-phenol

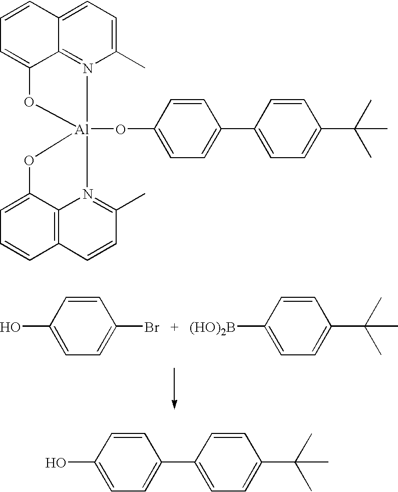

### 1a Preparation of Phenolic Ligand

A degassed solution of K₂CO₃ (31.52 g, 22.8 mmol) in H₂O (400 mL) was added to a mixture of 4-bromophenol (5.19 g, 30 mmol), 4-(t-butylphenyl)boronic acid (10.38 g, 66 mmol) and Pd(PPh₃)₄ (2.00 g, 1.72 mmol) in monoglyme (400 mL) and then heated to 80° C. for two days. Upon cooling, the mixture was diluted with diethylether and 1M HCl (˜100 mL) was added. After neutralization with a saturated solution of NaHCO₃, the organic layer was separated and dried over MgSO₄. Upon evaporation of the solvent a yellow solid was obtained which was purified by chromatography on silica (hexane) to obtain the desired product as a white powder (13.1 g, 96%).

### 1b Preparation of the Aluminum Complex Containing Ligand Prepared in 1a

In glove box, 1.60 g of quinaldine (2-methyl-8-hydroxyquinoline) was dissolved into 25 mL toluene with stirring in a 300 mL RB flask. 2.75 mL of 1.9M triethylaluminum in toluene solution (Aldrich) was added by syringe with rapid stirring. Addition must be done slowly to prevent overflow, as solution foams. The solution became cloudy and eventually a dense yellow fibrous ppt formed. The solution was refluxed in a heating mantle and the solid redissolved as the solution became clear yellow. 1.13 g of the ligand prepared in 1a was added as a solution in 5 mL toluene to yield a pale orange clear solution after heat and stirring. The solution was refluxed for 30 mins.

Addition of hexanes leads to a ppt of white crystals of the desired product in moderate yield of ˜1.4 g. The crystals were collected by filtration and suction dried. The resulting material is blue photoluminescent and has the 1-H nmr in methylene chloride shown. Product Tg: 121° C. The material was sublimed in high vacuum prior to device fabrication.

### Example 2 Preparation of an Aluminum Complex of 6-(4-t-butylphenyl)-naphth-2-ol

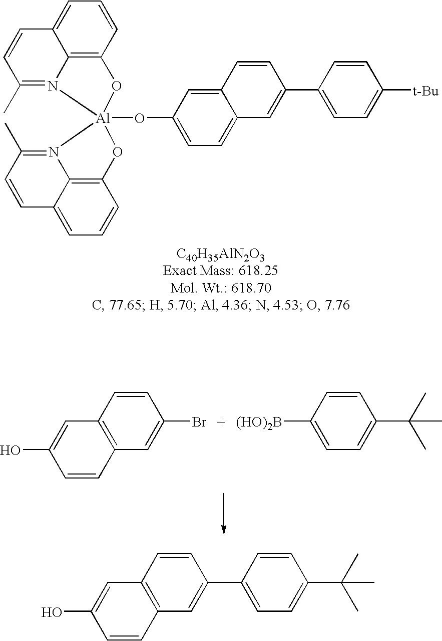

### 2a Preparation of the Phenolic Ligand

A degassed solution of K₂CO₃ (8.2 g, 5.9 mmol) in H2O (100 mL) was added to a mixture of 6-bromo-2-naphthol (3.43 g, 1.54 mmol), 4-(t-butylphenyl)boronic acid (3.21 g, 1.80 mmol) and Pd(PPh₃)₄ (0.89 g, 0.77 mmol) in monoglyme (100 mL) and then heated to 80° C. for two days. Upon cooling, the mixture was diluted with diethylether and 1M HCl (˜20 mL) was added. After neutralization with a saturated solution of NaHCO₃, the organic layer was separated and dried over MgSO₄. Upon evaporation of the solvent a yellow solid was obtained which was purified by chromatography on silica (hexane) to obtain the desired product as a tan powder (3.8 g, 89%).

### 2b Preparation of Aluminum Complex Containing Ligand Prepared in 2a

In a glove box, 1.60 g of quinaldine (2-methyl-8-hydroxyquinoline) was dissolved into 25 mL toluene with stirring in a 300 mL RB flask. 2.65 mL of 1.9M triethylaluminum in toluene solution (Aldrich) was added via syringe and rapid stirring. There is much foaming and addition must be done slowly to prevent overflow. The solution becomes cloudy and eventually a dense yellow fibrous ppt forms. The solution is brought to reflux in a heating mantle and the solid redissolves as the solution becomes clear yellow. 1.4 g of the ligand prepared in 2a was added as a solid with heat and stirring. Reflux with an air condensor in the glove box for 2 hrs then cool.

The initial solution is yellow orange and clear. As the solution cools there is a slight ppt and hexanes is added to enhance pptn. The desired product material crystallizes from hot toluene as a pale yellow solid when a little hexanes is added. Yield 2.9 g. 1-H nmr in methylene chloride reveals the expected product contaminated with a minor second isomer. Melting Point 264° C., Tg 131° C. The material was sublimed in high vacuum prior to device fabrication.

### Example 3 Preparation of an Aluminum Complex of 4-(3,5-dimethylphenyl)-phenol

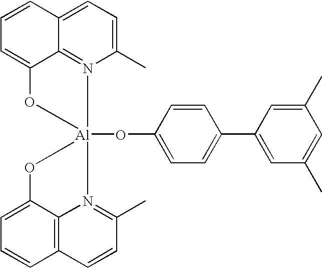

### 3a Preparation of the Phenolic Ligand

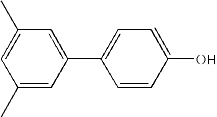

A mixture of 4.9 g of 4-Br-phenol, 4.5 g of 3,5-dimethylphenylboronic acid, 19.5 g of potassium carbonate, 0.3 g of tetrakis(triphenylphosphine)-palladium, 150 mL of water and 150 mL of 1,2-dimethoxyethane was refluxed (85-90° C.) for 12 hrs. The reaction mixture was diluted with 600 mL of water, extracted with dichloromethane (75 ml×3), and the combined organic layer was dried over MgSO₄. Solvent was removed under vacuum and the residue was distilled under reduced pressure to give 1.5 g of 3a >95% purity, b.p. 125-133° C./0.1 mm Hg.

### 3b Preparation of the Aluminum Complex of Ligand Prepared in 3a

In a glove box, 1.6 g of quinaldine (2-methyl-8-hydroxyquinoline) was dissolved into 25 mL toluene with stirring in a 300 mL RB flask. 2.65 mL of 1.9M triethylaluminum in toluene solution (Aldrich) was added via syringe and with rapid stirring. The addition must be done slowly to prevent overflow. The solution becomes cloudy and eventually a dense yellow fibrous ppt forms. The solution is brought to reflux in a heating mantle and it becomes clear bright yellow. 1.0 g phenol prepared in 3a was added as a solution in 5 mL toluene and the solution was heated and stirred as a bright yellow solution. Reflux for 30 mins then cool and add methanol and hexanes. A pale yellow crystalline solid precipitates on standing and is collected by filtration, washed well with methanol and suction dried to yield a material having the 1-H nmr spectrum shown below. The material contains a small amount of a second isomeric material. Melting Point 265° C., Tg 105° C. The material was sublimed in high vacuum prior to device fabrication.

### Example 4 Preparation of an Aluminum Complex of 6-(3,5-dimethylphenyl)-naphth-2-ol

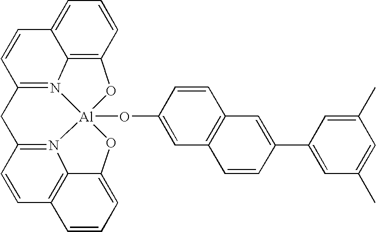

### 4a Preparation of the Phenolic Ligand

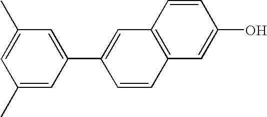

Similar to the synthesis given in example 3a, 11.6 g of 6-bromonaphthol, 8.2 g of 3,5-dimethylphenylboronic acid, 300 mL of deionized water, 36 g of potassium carbonate, 0.3 g of tetrakis(triphenylphosphine)palladium, and 250 mL of 1,2-dimethoxyethane was refluxed (85-90° C.) for 12 hr. The reaction mixture was then diluted with 600 mL of 10% hydrochloric acid, extracted with dichloromethane (75 ml×3), and the combined organic layer was washed with water (300 mL) and dried over MgSO₄. The solvent was removed under vacuum and the residue was redissolved into hot hexane and the hot solution was put through a short silica gel plug. The solvent was removed and the solid residue was dried under vacuum to afford 6 g (65%) of 4a. ¹H NMR (CDCl₃): 2.50 (6H, s), 7.10 (1H, s), 7.22 (2H, m), 7.38 (2H, s), 7.75 (2H, s), 7.85 (1H, d), 8.05 (1H, s) ppm.

### 4b Preparation of the Aluminum Complex of Ligand Prepared in 4a

In a glove box, 3.2 g of quinaldine (2-methyl-8-hydroxyquinoline) was dissolved into 25 mL toluene with stirring in a 300 mL RB flask. 5.3 mL of 1.9M triethylaluminum in toluene solution (Aldrich) was added via syringe and with rapid stirring. There is much foaming and addition must be done slowly to prevent overflow. The solution becomes cloudy and eventually a dense yellow fibrous ppt forms. The solution is brought to reflux in a heating mantle and it becomes clear yellow. 2.5 g phenol prepared in 4a was added as a solution in 5 mL toluene and then heated and stirred as a pale orange solution. Reflux for 30 mins and then cool to room temperature whereupon a very pale yellow crystalline precipitate begins to deposit. The crystals were collected by filtration and washed well with methanol and suction dried. Yield of 5.3 g of pale yellow powder. Tg 117° C. The solid is blue photoluminescent and has the 1-H nmr spectrum shown below

### Example 5 Preparation of an Aluminum Complex of 6-(3,5-bis-trifluoromethyl-phenyl)-naphth-2-ol

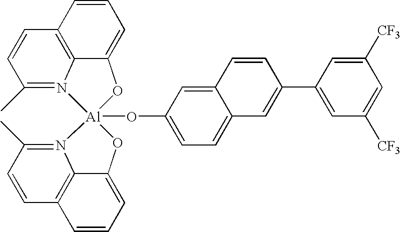

### 5a Preparation of the Phenolic Ligand

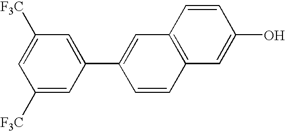

6-bromo-2-naphthol 6.1 g, 0.0273 mol; 3,5-trifluoromethylphenylboronic acid 7.6 g, 0.0295 mol; potassium carbonate 18.6 g, 0.1346 mol; water 180 mL; ethyleneglycoldimethylether 180 mL were combined and sparged with nitrogen for 45 minutes. Tetrakistriphenylphosphine Pd(0) (1.2 g, 0.001 mol) was then added and the mixture was refluxed overnight. Upon cooling to room temperature 500 mL of acidic water was added and extracted with dichloromethane. The organic layer was preabsorbed onto silica gel and eluted down a silica column with hexanes 80%/ethylacetate 20%. The eluted organic material was concentrated and taken up in ethanol and stirred with activated carbon, filtered and concentrated_to precipitate the desired phenolic ligand in 75% yield.

### 5b Preparation of the Aluminum Complex of Ligand Prepared in 5a

In a glove box, 3.2 g of quinaldine (2-methyl-8-hydroxyquinoline) was dissolve into 25 mL toluene with stirring in a 300 mL RB flask. 5.3 mL of 1.9M triethylaluminum in toluene solution (Aldrich) was added via syringe with rapid stirring. There is much foaming and addition must be done slowly to prevent overflow. The solution becomes cloudy and eventually a dense yellow fibrous ppt forms. The solution is brought to reflux in a heating mantle and it becomes clear yellow. 3.6 g phenol prepared in 5a was added as a solution in 5 mL toluene then heated and stirred as a pale orange solution. Reflux for 30 mins then cool and add methanol. Evaporation and additional methanol additions deposits a pale yellow crystalline material which was collected by filtration, washed well with methanol and suction dried. 5.5 g of pale yellow crystals were collected with MPt 247° C. and Tg 114° C. The crystals were blue green photoluminescent and highly soluble in toluene, >2.8 wt %. 1-H nmr spectra of the material in methylene chloride are shown below

### Example 6 Preparation of an Aluminum Complex of 4-(4-trifluoromethylphenyl)-phenol

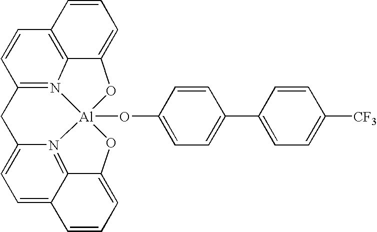

### 6a Preparation of the Phenolic Ligand

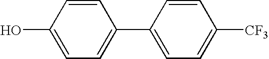

Under an atmosphere of nitrogen, 4-bromophenol (5.00 g, 2.91×10⁻² mol), 4-CF₃-1-phenyl boronic acid (6.075 g, 3.20×10⁻² mol), KF (5.57 g, 9.60×10⁻² mol), Pd₂(dba)₃ (0.133 g, 1.45×10⁻⁴ mol) were mixed in THF (100 mL). After a few minutes of stirring a THF (10 mL) solution of P(^{t}Bu)₃ (0.059 g, 2.91×10⁻⁴ mol). The resulting mixture was stirred at room temperature for 48 hours. Dilution of this solution with 200 mL of Et₂O followed by filtration through a silica plug and evaporation of the volatiles yielded a dark brown thick oil. Upon addition of hexane to this oil, a light brown powder formed, which was further purified by column chromatography (1:5 EtOAc/hexane) to give a white powder. Yield=4.86 g (70%). The ¹H NMR (in CD₂Cl₂) spectrum of phenol 6a is shown below.

### 6b Preparation of the Aluminum Complex of Ligand Prepared in 6a

In a glove box, 3.2 g of quinaldine (2-methyl-8-hydroxyquinoline) was dissolve into 25 mL toluene with stirring in a 300 mL RB flask. 5.3 mL of 1.9M triethylaluminum in toluene solution (Aldrich) was added via syringe with rapid stirring. There is much foaming and addition must be done slowly to prevent overflow. The solution becomes cloudy and eventually a dense yellow fibrous ppt forms. The solution is brought to reflux in a heating mantle and it becomes clear yellow. 2.4 g phenol prepared in 6a was added as a solution in 5 mL toluene then heated and stirred as a pale orange solution. Reflux for 30 mins then cool and add methanol. Evaporation and additional methanol additions deposits an off-white crystalline material which was collected by filtration, washed well with methanol and suction dried. 2.5 g of white crystals were collected with MPt 202° C. and Tg 92° C. The crystals were blue green photoluminescent and highly soluble in toluene, >2.8 wt %. 1-H nmr spectra of the material in methylene chloride are shown below. The material was sublimed under high vacuum prior to device evaluations.

### Example 7 Preparation of an Aluminum Complex of 4-(3-cyanophenyl)-phenol

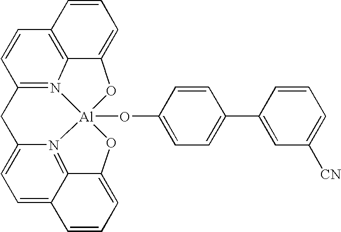

### 7a Preparation of the Phenolic Ligand

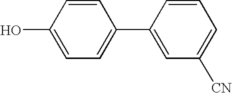

Under an atmosphere of nitrogen, 4-bromophenol (5.30 g, 3.08×10⁻² mol), 3-CN-1-phenyl boronic acid (4.986 g, 3.39×10⁻² mol), KF (5.91 g, 1.02×10⁻¹ mol), Pd₂(dba)₃ (0.141 g, 1.54×10⁻⁴ mol) were mixed in THF (100 mL). After a few minutes of stirring a THF (10 mL) solution of P(^{t}Bu)₃ (0.062 g, 3.08×10⁻⁴ mol). The resulting mixture was stirred at room temperature for 48 hours. Dilution of this solution with 200 mL of Et₂O followed by filtration through a silica plug and evaporation of the volatiles yielded a dark brown thick oil. Upon addition of hexane to this oil, a light brown powder formed, which was further purified by column chromatography (1:5 EtOAc/hexane) to give a white powder. Yield=3.9 g (65%). The ¹H NMR (in CD₂Cl₂) spectrum of phenol 7a is shown below.

### 7b Preparation of the Aluminum Complex of Ligand Prepared in 7a

In a glove box, 3.2 g of quinaldine (2-methyl-8-hydroxyquinoline) was dissolve into 25 mL toluene with stirring in a 300 mL RB flask. 5.3 mL of 1.9M triethylaluminum in toluene solution (Aldrich) was added via syringe with rapid stirring. There is much foaming and addition must be done slowly to prevent overflow. The solution becomes cloudy and eventually a dense yellow fibrous ppt forms. The solution is brought to reflux in a heating mantle and it becomes clear yellow. 1.96 g phenol prepared in 7a was added as a solution in 5 mL toluene then heated and stirred as a pale orange solution. Reflux for 30 mins then cool and add methanol. Evaporation and additional methanol additions deposits a cream colored crystalline material which was collected by filtration, washed well with methanol and suction dried. 5.0 g of cream crystals were collected with MPt 219° C. and Tg 106° C. The crystals were blue green photoluminescent and quite soluble in toluene, ˜1.6 wt %. 1-H nmr spectra of the material in methylene chloride are shown below. The material was sublimed in high vacuum prior to device evaluation

### Example 8 Preparation of an Aluminum Complex of 4-(4-cyanophenyl)-phenol

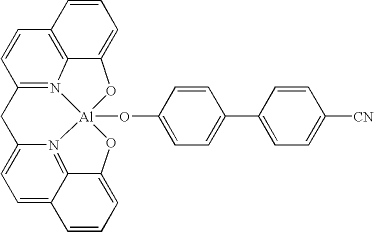

### 8a Preparation of the Phenolic Ligand

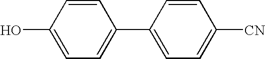

Under an atmosphere of nitrogen, 4-bromophenol (5.30 g, 3.08×10⁻² mol), 4-CN-1-phenyl boronic acid (4.986 g, 3.39×10⁻² mol), KF (5.91 g, 1.02×10⁻¹ mol), Pd₂(dba)₃ (0.141 g, 1.54×10⁻⁴ mol) were mixed in THF (100 mL). After a few minutes of stirring a THF (10 mL) solution of P(^{t}Bu)₃ (0.062 g, 3.08×10⁻⁴ mol). The resulting mixture was stirred at room temperature for 48 hours. Dilution of this solution with 200 mL of Et₂O followed by filtration through a silica plug and evaporation of the volatiles yielded a dark brown thick oil. Upon addition of hexane to this oil, a light brown powder formed, which was further purified by column chromatography (1:5 EtOAc/hexane) to give a white powder. Yield=3.3 g (55%). The ¹H NMR (in CD₂Cl₂) spectrum of phenol 8a is shown below.

### 8b Preparation of the Aluminum Complex of Ligand Prepared in 8a

In a glove box, 3.2 g of quinaldine (2-methyl-8-hydroxyquinoline) was dissolve into 25 mL toluene with stirring in a 300 mL RB flask. 5.3 mL of 1.9M triethylaluminum in toluene solution (Aldrich) was added via syringe with rapid stirring. There is much foaming and addition must be done slowly to prevent overflow. The solution becomes cloudy and eventually a dense yellow fibrous ppt forms. The solution is brought to reflux in a heating mantle and it becomes clear yellow. 1.96 phenol prepared in 8a was added as a solution in 5 mL toluene then heated and stirred as a pale orange solution. Reflux for 30 mins then cool and add methanol. Evaporation and additional methanol additions deposits a cream colored crystalline material which was collected by filtration, washed well with methanol and suction dried. 1.6 g of cream colored crystals were collected with MPt 180° C. and Tg 123° C. The crystals were blue green photoluminescent and quite soluble in toluene, ˜1.6 wt %. 1-H nmr spectra of the material in methylene chloride are shown below. The material was sublimed in high vacuum prior to device evaluations.

### Example 9

### Device Fabrication and Characterization Data

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with 1400 Å of ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen. Immediately before device fabrication the cleaned, patterned ITO substrates were treated with O₂ plasma for 5 minutes. Immediately after cooling, an aqueous dispersion of Buffer 1 was spin-coated over the ITO surface and heated to remove solvent. After cooling, the substrates were then spin-coated with a solution of Hole Transport 1, and then heated to remove solvent. After cooling the substrates were spin-coated with the emissive layer solution, and heated to remove solvent. The substrates were masked and placed in a vacuum chamber. A ZrQ layer was deposited by thermal evaporation, followed by a layer of LiF. Masks were then changed in vacuo and a layer of Al was deposited by thermal evaporation. The chamber was vented, and the devices were encapsulated using a glass lid, dessicant, and UV curable epoxy.

In Example 9.1, the host was a mixture of the material of Example 3 and Host A. The emitter was Red emitter 2.

In Example 9.2, the host was a mixture of the material of Example 5 and Host B. The emitter was Red emitter 1.

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The power efficiency is the current efficiency divided by the operating voltage. The unit is lm/W.

The materials used in device fabrication are listed below:

Buffer 1 was an aqueous dispersion of poly(3,4-dioxythiophene) and a polymeric fluorinated sulfonic acid. The material was prepared using a procedure similar to that described in Example 3 of published U.S. patent application no. 2004/0254297.
Hole Transport 1 was a crosslinkable polymeric hole transport material.

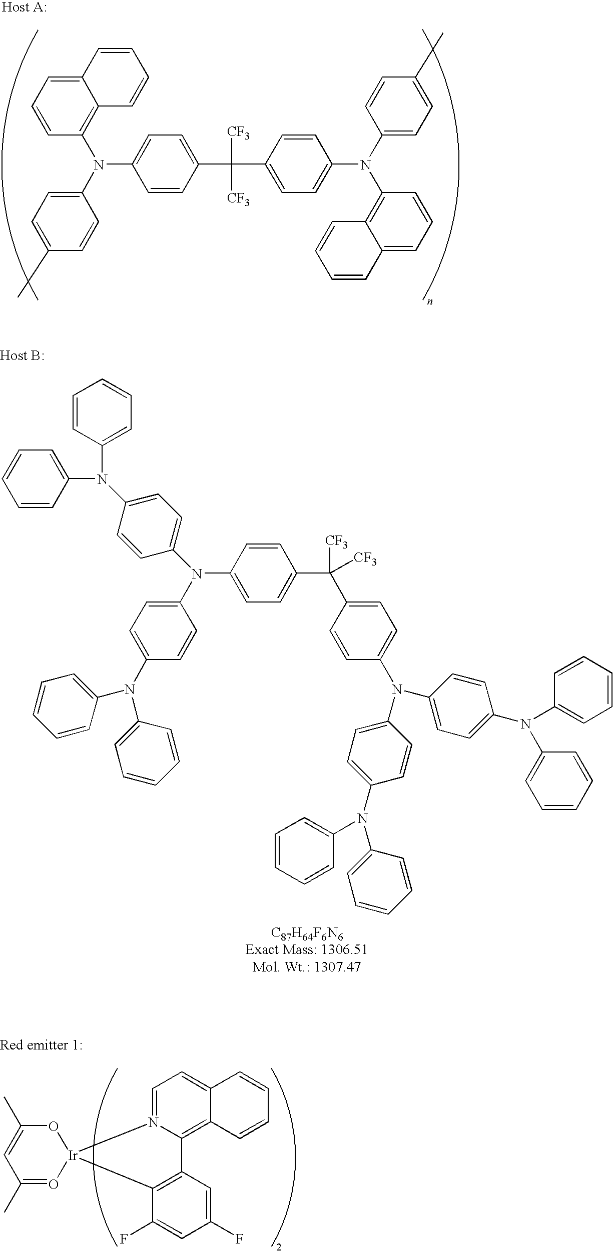

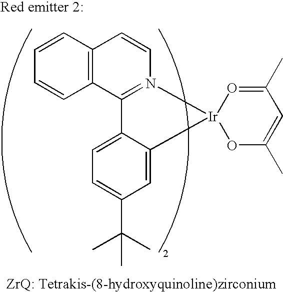

TABLE 9.1
Device characterization data

| | Current efficiency | Power efficiency | Color coordinates, |
|---|---|---|---|
| Example | at 500 nits, cd/A | at 500 nits, lm/W | (x, y) |
| 9.1 | 8.3 | 4.4 | (0.68, 0.31) |
| 9.2 | 4.9 | 2.2 | (0.65, 0.34) |

### Example 10

### Device Fabrication and Characterization Data

OLED devices were fabricated by the thermal evaporation technique. The base vacuum for all of the thin film deposition was in the range of 10⁻⁸ torr. Patterned indium tin oxide coated glass substrates from Thin Film Devices, Inc were used. These ITO's are based on Corning 1737 glass coated with 1400 Å ITO coating, with sheet resistance of 30 ohms/square and 80% light transmission. The patterned ITO substrates were then cleaned ultrasonically in aqueous detergent solution. The substrates were then rinsed with distilled water, followed by isopropanol, and then degreased in toluene vapor. Immediately after cleaning, an aqueous dispersion of Buffer 1 was spin-coated over the ITO surface and heated to remove solvent.

The substrate was then loaded into the vacuum chamber and multiple layers of thin films were then deposited sequentially onto the buffer layer by thermal evaporation. Patterned layers of LiF and Al were deposited through a mask. The completed OLED device was then taken out of the vacuum chamber, encapsulated with a cover glass using epoxy, and characterized. The device layers are given in Table 10.1 below. The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The power efficiency is the current efficiency divided by the operating voltage. The unit is lm/W.

The materials used in device fabrication are listed below:

NPB: N,N′-Bis(naphthalen-1-yl)-N,N′-bis-(phenyl)benzidine

### TDATA: 4,4′,4″-Tris-(N,N-diphenyl-amino)-triphenylamine

TABLE 10.1
Device summary:

| Example | Hole transport | Host | Emitter |
|---|---|---|---|
| 10.1 | TDATA | Material of Example 2 | Red emitter 1 |
| 10.2 | NPB | Material of Example 3 | Red emitter 2 |
| 10.3 | NPB | Material of Example 4 | Red emitter 2 |
| 10.4 | TDATA | Material of Example 5 | Red emitter 1 |
| 10.5 | NPB | Material of Example 7 | Red emitter 1 |

TABLE 10.2
Device characterization data

| | Peak | Current | Power | Color |
|---|---|---|---|---|
| | efficiency, | efficiency at 500 | efficiency at | coordinates, |
| Example | cd/A | nits, cd/A | 500 nits, lm/W | C.I.E. (x, y) |
| 10.1 | 16 | 9.2 | 4.8 | (0.65, 0.35) |
| 10.2 | 16 | 10 | 3.8 | (0.67, 0.33) |
| 10.3 | 12 | 8.9 | 4.2 | (0.67, 0.33) |
| 10.4 | 11.6 | 11.6 | 2.6 | (0.65, 0.35) |
| 10.5 | 15 | 6 | 2.5 | (0.65, 0.35) |

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Many aspects and embodiments have been described above and are merely exemplary and not limiting. After reading this specification, skilled artisans appreciate that other aspects and embodiments are possible without departing from the scope of the invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

1. A ligand for a metal, the ligand comprising Formula I or II: 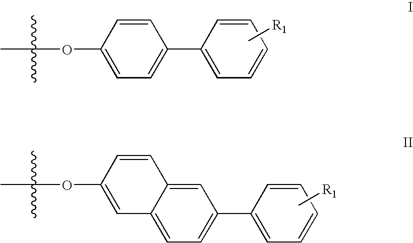
wherein R₁ is alkyl or aryl.

2. The ligand of claim 1, wherein R₁ is in the para-position.

3. The ligand of claim 1, wherein R₁ is a C4-C5 alkyl.

4. The ligand of claim 1, wherein R₁ is t-butyl.

5. The ligand of claim 1, wherein R₁ is aryl.

6. An organometallic complex comprising formula:
[Y]ₙMZ
wherein:
n is 1, 2, or 3;
M is a metal in a +2, +3, or +4 oxidation state;
Y is a ligand comprising 8-hydroxyquinoline or alkyl-substituted 8-hydroxyquinoline at each occurrence; and
Z is a ligand comprising Formula I or II: 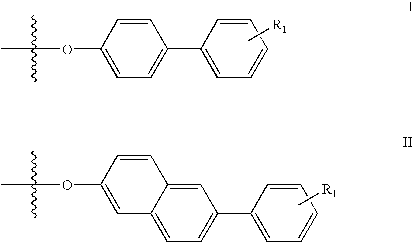
wherein R₁ is alkyl or aryl.

7. The complex of claim 6, wherein M is Al, Zn, Zr, or Ga.

8. The complex of claim 6, wherein the alkyl-substituted 8-hydroxyquinoline is substituted at the 2 position.

9. The complex of claim 6, wherein the alkyl-substituted 8-hydroxyquinoline is 2-methyl-8-hydroxyquinoline.

10. The complex of claim 6 that is:

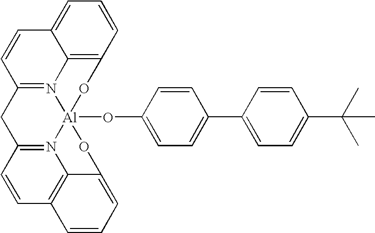

11. The complex of claim 6 that is:

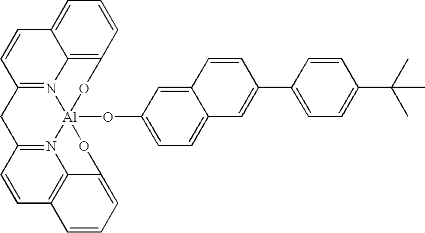

12. An organometallic complex having the formula:
MYₙZ
wherein:
n is 1, 2, or 3;
M is a metal in a +2, +3, or +4 oxidation state;
Y is selected from 8-hydroxyquinolate and substituted 8-hydroxyquinolate; and
Z is a compound of formula III or IV: 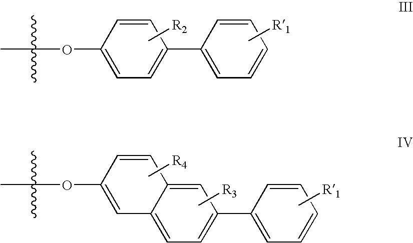
wherein:
R′₁ is one or more solvent-solubilizing or Tg enhancing groups;
R₂, R₃, and R₄ are independently one or more selected from the group consisting of H, alkyl, substituted alkyl, aryl, substituted aryl, F, CN, a solvent-solubilizing group, and a Tg enhancing group.

13. The complex of claim 12, wherein M is Al, Zn, Zr, In, or Ga.

14. The complex of claim 12, wherein R′₁ is an alkyl group.

15. The complex of claim 12, wherein R′₁ is cyano, alkyl, fluoroalkyl, aryl, fluoroaryl, alkylaryl, alkoxy, aryloxy, fluoroalkoxy, or fluoroaryloxy, or their hetero-analogs.

16. The complex of claim 12, wherein R′₁ is phenyl, fluorophenyl, alkylphenyl, fluoroalkylphenyl, alkoxy phenyl, or fluoroalkoxyphenyl.

17. A composition comprising the complex of claim 6 and at least one solvent, processing aid, charge transporting material, or charge blocking material.

18. An organic electronic device comprising an active layer and the complex of claim 6.

19. The device of claim 18, wherein the active layer is a photoactive layer, and the complex is in or adjacent to the photoactive layer.

20. An article useful in the manufacture of an organic electronic device, comprising the complex of claim 6.
